(19) United States
(12) Patent Application Publication
(10) Pub. No.: US 2008/0103199 A1
(43) Pub. Date: May 1, 2008

(54) TREATMENT OF PERVASIVE DEVELOPMENTAL DISORDERS

(76) Inventor: Magali HAAS, Corinth, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/872,177

(22) Filed: Oct. 15, 2007

Related U.S. Application Data
(60) Provisional application No. 60/863,595, filed on Oct. 31, 2006.

(51) Int. Cl. A61K 31/27; A61P 43/00

(52) U.S. Cl. 514/483; 514/485

(57) ABSTRACT

The present invention is a method for the treatment of Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) comprising administering to a subject in need thereof a therapeutically effective amount of one or more carbamate compounds of Formula 1 and/or Formula 2 as herein defined and shown below.

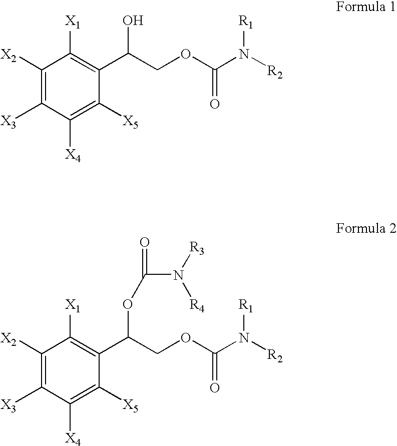

The present invention is directed to a method for the treatment of Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS), which includes mono-therapy and alternatively, co-therapy with at least one additional psychoactive medication.

This application claims the benefit under 35 U.S.C. 119(e) of U.S. Provisional application Ser. No. 60/863,595 filed Oct. 31, 2006. The complete disclosure of the aforementioned related U.S. patent application is hereby incorporated herein by reference for all purposes.

### FIELD OF THE INVENTION

The present invention is directed to the use of certain carbamate compounds for the treatment of patients with Pervasive Developmental Disorders including Autism.

### BACKGROUND OF THE INVENTION

The Pervasive Developmental Disorders (PDDs), are a category of neurological disorders characterized by severe and pervasive impairment in several areas of development, including social interaction and communications skills (DSM-IV-TR). The five disorders under PDD are Autistic Disorder, Asperger's Disorder, Childhood Disintegrative Disorder (CDD), Rett's Disorder, and PDD-Not Otherwise Specified (PDD-NOS). Specific diagnostic criteria for each of these disorders can be found in the Diagnostic & Statistical Manual of Mental Disorders (DSM-IV-TR) as distributed by the American Psychiatric Association (APA).

The most common of the Pervasive Developmental Disorders, autism affects an estimated 1 in approximately 200 births. Indeed, as of 2003-2004, as many as 1.5 million Americans are believed to have some form of autism. Such a number is on the rise inasmuch as, based on statistics from the U.S. Department of Education and other governmental agencies; autism is growing at a rate of 10-17 percent per year. At these rates, the ASA estimates that the prevalence of autism could easily reach 4 million Americans in the next decade.

Autism is a complex developmental disability that interferes with, among other things, the normal development of the brain in the areas of social interaction and communication skills. It typically appears during the first three years of life and is the result of a neurological disorder that affects the functioning of the brain. Typically, autistic children and adults have difficulties in verbal and non-verbal communication, social interactions, and leisure or play activities.

The overall incidence of autism is, for the most part, globally consistent. Indeed, autism knows no racial, ethnic, or social boundaries, and family income, lifestyle, and educational levels do not affect the chance of autism's occurrence. However, it has been found to be four times more prevalent in boys than girls.

Since being first described by Dr. Leo Kanner in 1943, the understanding of autism has grown tremendously. Although autism is defined by a certain set of behaviors, it is a spectrum disorder in that its symptoms and characteristics can be present in a wide variety of combinations, from mild to severe. Therefore, autistic children and adults can exhibit any combination of the behaviors in any degree of severity. Two individuals, both with the same diagnosis, may have varying skills and display very different actions. Those only mildly affected may exhibit slight delays in language or communication and may face greater challenges in social interactions. For example, one may have difficulty initiating and/or maintaining a conversation. Communication by autistic children or adults is often displayed as talking at others (for example, a monologue on a favorite subject that continues despite attempts by others to interject comments).

Autism seems to cause those affected by it to process and respond to information in unique ways. In some individuals with PDDs including Autism, aggressive and/or self-injurious behavior may exist. The following traits, as identified by the ASA, may also be present in persons with autism: insistence on sameness or resistance to change; difficulty in expressing needs; (i.e. uses gestures or pointing instead of words); repeating words or phrases in place of normal, responsive language; laughing, crying, showing distress for reasons not apparent to others; preferring to be alone or aloof manner; tantrums; difficulty in mixing with others; may not want to cuddle or be cuddled; little or no eye contact; unresponsive to normal teaching methods; sustained odd play; spins objects; inappropriate attachments to objects; apparent over-sensitivity or under-sensitivity to pain; no real fears of danger; marked physical over-activity or extreme under-activity; uneven gross/fine motor skills; and/or nonresponsivness to verbal cues (i.e. acts as if deaf although hearing tests in normal range).

For individuals with autism, sensory integration problems are common. In particular, their senses may be either over- or under-active. The fuzz of a kiwi may actually be experienced as painful; a sweet, fruity smell may cause a gagging reflex. Some children or adults with autism are particularly sensitive to sound, so that even the most ordinary daily noises are painful.

Although there is no single known cause for autism, it is generally accepted that it is caused by abnormalities in brain structure or function. The shape and structure of the brain in autistic versus non-autistic children show differences when brain scans are viewed. Currently the link between heredity, genetics and medical problems are being investigated by researchers, as well as a number of other theories. The theory of a genetic basis of the disorder is supported by the fact that, in many families, there appears to be a pattern of autism or related disabilities. While no one gene has been identified as causing autism, researchers are searching for irregular segments of genetic code that autistic children may have inherited. While researchers have not yet identified a single trigger that causes autism to develop, it also appears that some children are born with a susceptibility to autism.

Other researchers are investigating the possibility that under certain conditions, a cluster of unstable genes may interfere with brain development resulting in autism. Still other researchers are investigating problems during pregnancy or delivery as well as environmental factors such as viral infections, metabolic imbalances, and exposure to environmental chemicals.

According to the ASA, autism tends to occur more frequently than expected among individuals who have certain medical conditions, including Fragile X syndrome, tuberous sclerosis, congenital rubella syndrome, and untreated phenylketonuria (PKU). Some harmful substances ingested during pregnancy also have been associated with an increased risk of autism.

Early in 2002, The Agency for Toxic Substances and Disease Registry (ATSDR) prepared a literature review of hazardous chemical exposures and autism found no compelling evidence for an association; however, there was very limited research and more needs to be done.

Whatever the cause, parents can rest assured that autism is not caused by bad parenting. Children with autism and PDD are either born with the disorder or with the potential to develop it. No known psychological factors in the development of the child have been shown to cause autism.

Notwithstanding the foregoing, and to the best of applicant's knowledge, there is no cure for autism. There are, however, a number of medications, developed for other conditions, which have been found to be somewhat helpful in treating a limited number of the symptoms and behaviors frequently found in individuals with autism, such as hyperactivity, impulsivity, attention difficulties, and anxiety. Examples of medications used to treat symptoms associated with autism include: serotonin re-uptake inhibitors (e.g. clomipramine (Anafranil), fluvoxamine (Luvox) and fluoxetine (Prozac)) that have been effective in treating depression, obsessive-compulsive behaviors, and anxiety that are sometimes present in autism. Studies have shown that they may reduce the frequency and intensity of repetitive behaviors, and may decrease irritability, tantrums and aggressive behavior. Some children have shown improvements in eye contact and responsiveness. Other drugs, such as Elavil, Wellbutrin, Valium, Ativan and Xanax, require more studies to be done but may have a role in reducing behavioral symptoms.

Over the past 35 years, the most widely studied psychopharmacologic agents in autism have been anti-psychotic medications. Originally developed for treating schizophrenia, these drugs have been found to decrease hyperactivity, stereotypic behaviors, withdrawal and aggression in autistic children. Four that have been approved by the FDA are clozapine (Clozaril), risperidone (Risperdal), olanzapine (Zyprexa) and quetiapine (Seroquel). However, only risperidone has been investigated in a controlled study of adults with autism. Unfortunately, like the antidepressants, these drugs all have adverse side effects, including, but not limited to, sedation. In addition, anti-psychotic medications can induce temporary and/or persisting movement disorders including Tardive Dyskinesia that may be permanent.

Stimulants, such as Ritalin, Adderall, and Dexedrine, used to treat hyperactivity in children with ADHD have also been prescribed for children with autism. Although few studies have been done, they may increase focus, and decrease impulsivity and hyperactivity in autism, particularly in higher-functioning children. Unfortunately, adverse behavioral side effects are often observed.

While many of the above-identified medications do appear to be somewhat helpful in treating a limited number of the symptoms and behaviors frequently found in individuals with autism, a wide variety of side effects are associated with such medications.

Therefore there is a need to provide additional methods for treating the behavioral manifestations of autism via administering an effective amount of a medication that does not have the potential side effects of the above described treatments.

### SUMMARY OF THE INVENTION

The present invention is directed to a method for the treatment of Pervasive Developmental Disorders (PDDs) including the five disorders listed under PDD in the DSM-IV-TR, i.e., Autistic Disorder, Asperger's Disorder, Childhood Disintegrative Disorder (CDD), Rett's Disorder, and Pervasive Developmental Disorders-Not Otherwise Specified (PDD-NOS), comprising administering to a subject in need thereof a therapeutically effective amount of a composition that comprises at least one compound of Formula 1 or Formula 2:

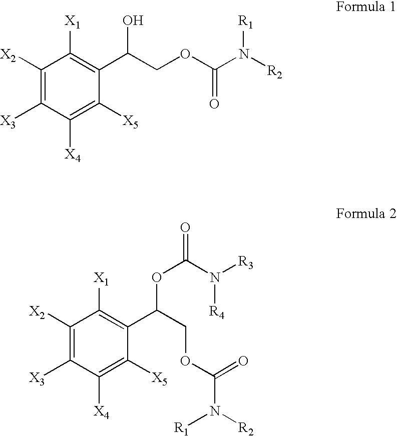

or a pharmaceutically acceptable salt or ester form thereof,
wherein
R₁, R₂, R₃ and R₄ are independently hydrogen or C₁-C₄ alkyl,
wherein
C₁₋C₄alkyl is substituted or unsubstituted with phenyl, and
wherein
phenyl is substituted or unsubstituted with up to five substituents independently selected from; halogen, C₁₋C₄ alkyl, C₁₋C₄ alkoxy, nitro, cyano and amino
wherein amino is optionally mono or disubstituted with C₁₋C₄ alkyl,
and X₁, X₂, X₃, X₄ and X₅ are independently hydrogen, fluorine, chlorine, bromine or iodine.

Embodiments of the present invention include a compound of Formula 1 or Formula 2 wherein X₁, X₂, X₃, X₄ and X₅ are independently selected from;

hydrogen, fluorine, chlorine, bromine or iodine.

In certain embodiments, X₁, X₂, X₃, X₄ and X₅ are independently selected from hydrogen or chlorine.

In other embodiments, X₁ is selected from fluorine, chlorine, bromine or iodine. In another embodiment, X₁ is chlorine, and X₂, X₃, X₄ and X₅ are hydrogen. In another embodiment, R₁, R₂, R₃ and R₄ are hydrogen.

The present invention provides enantiomers of Formula 1 or Formula 2 for treating Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) in a subject in need thereof. In certain embodiments, a compound of Formula 1 or Formula 2 will be in the form of a single enantiomer thereof. In other embodiments, a compound of Formula 1 or Formula 2 will be in the form of an enantiomeric mixture in which one enantiomer predominates with respect to another enantiomer.

In another aspect, one enantiomer predominates in a range of from about 90% or greater. In a further aspect, one enantiomer predominates in a range of from about 98% or greater.

The present invention also provides methods comprising administering to the subject a prophylactically or therapeutically effective amount of a composition that comprises at least one compound of Formula 1 or Formula 2 wherein R₁, R₂, R₃ and R₄ are independently selected from hydrogen or C₁-C₄ alkyl; and X₁, X₂, X₃, X₄ and X₅ are independently selected from hydrogen, fluorine, chlorine, bromine or iodine.

Exemplifying the invention is a method of treating Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) comprising administering to a subject in need thereof a therapeutically effective amount of any of the compounds or pharmaceutical compositions described above.

### BRIEF DESCRIPTION OF THE FIGURES

FIG. 1: The DSR apparatus

FIG. 2: Effect of treatment of submissive rats with Compound # 7 and fluoxetine on time spent at feeder

FIG. 3: Effect of Compound # 7 and fluoxetine on dominance level in pairs of rats.

FIG. 4: Effect of Treatment of Dominant Rats with COMPOUND #7 and Lithium on Time Spend at the Feeder.

FIG. 5: Effect of COMPOUND #7 and Lithium on Dominance Level in Pairs of Rats

### DETAILED DESCRIPTION OF THE INVENTION

The present invention is directed to methods for the treatment of Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) comprising administering to a subject in need thereof a therapeutically effective amount of a composition containing 2-phenyl-1,2-ethanediol monocarbomates and dicarbamates.

### The Carbamate Compounds of the Invention

Representative carbamate compounds according to the present invention include those having Formula 1 or Formula 2:

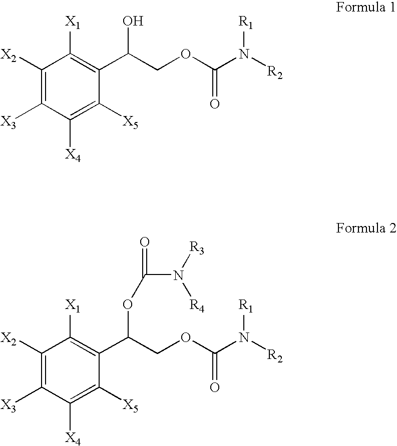

or a pharmaceutically acceptable salt or ester form thereof
wherein:
R₁, R₂, R₃ and R₄ are independently hydrogen or C₁-C₄ alkyl,
wherein
C₁₋C₄ alkyl is substituted or unsubstituted with phenyl, and
wherein
phenyl is substituted or unsubstituted with up to five substituents independently selected from; halogen, C₁₋C₄ alkyl, C₁₋C₄ alkoxy, nitro, cyano and amino
wherein amino is optionally mono or disubstituted with C₁₋C₄ alkyl,
and X₁, X₂, X₃, X₄ and X₅ are independently hydrogen, fluorine, chlorine, bromine or iodine.

“C₁-C₄ alkyl” as used herein refers to substituted or unsubstituted aliphatic hydrocarbons having from 1 to 4 carbon atoms. Specifically included within the definition of “alkyl” are those aliphatic hydrocarbons that are optionally substituted. In a preferred embodiment of the present invention, the C₁-C₄ alkyl is either unsubstituted or substituted with phenyl.

The term “phenyl”, as used herein, whether used alone or as part of another group, is defined as a substituted or unsubstituted aromatic hydrocarbon ring group having 6 carbon atoms. Specifically included within the definition of “phenyl” are those phenyl groups that are optionally substituted. For example, in a preferred embodiment of the present invention, the, “phenyl” group is either unsubstituted or substituted with halogen, C₁-C₄ alkyl, C₁-C₄ alkoxy, amino, nitro, or cyano.

In a preferred embodiment of the present invention, X₁ is fluorine, chlorine, bromine or iodine and X₂, X₃, X₄, and X₅ are hydrogen.

In another preferred embodiment of the present invention, X₁, X₂, X₃, X₄, and X₅ are, independently, chlorine or hydrogen.

In another preferred embodiment of the present invention, R₁, R₂, R₃, and R₄ are all hydrogen.

It is understood that substituents and substitution patterns on the compounds of the present invention can be selected by one of ordinary skill in the art to provide compounds that are chemically stable and that can be readily synthesized by techniques known in the art as well as the methods provided herein.

Representative 2-phenyl-1,2-ethanediol monocarbomates and dicarbamates include, for example, the following compounds:

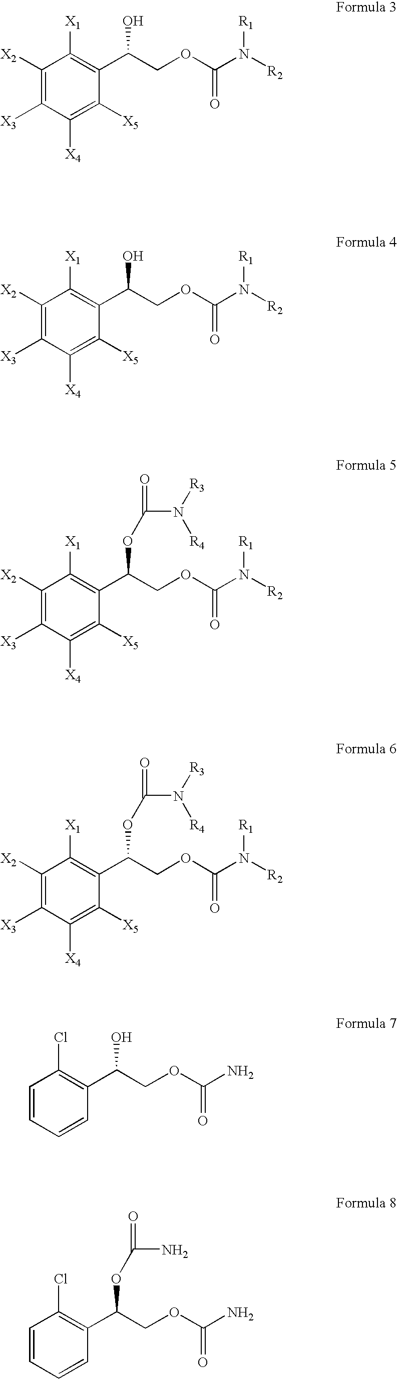

Suitable methods for synthesizing and purifying the carbamate compounds, including carbamate enantiomers, used in the methods of the present invention are well known to those skilled in the art. For example, pure enantiomeric forms and enantiomeric mixtures of 2-phenyl-1,2-ethanediol monocarbomates and dicarbamates are described in U.S. Pat. Nos. 5,854,283, 5,698,588, and 6,103,759, the disclosures of which are herein incorporated by reference in their entirety.

The present invention includes the use of isolated enantiomers of Formula 1 or Formula 2.

In one preferred embodiment, a pharmaceutical composition comprising the isolated S-enantiomer of Formula 1 is used to treat Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) in a subject in need thereof.

In another preferred embodiment, a pharmaceutical composition comprising the isolated R-enantiomer of Formula 2 is used to treat Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) in a subject in need thereof.

In another embodiment, a pharmaceutical composition comprising the isolated S-enantiomer of Formula 1 and the isolated R-enantiomer of Formula 2 can be used to treat Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) in a subject in need thereof.

The present invention also includes the use of mixtures of enantiomers of Formula 1 or Formula 2. In one aspect of the present invention, one enantiomer will predominate. An enantiomer that predominates in the mixture is one that is present in the mixture in an amount greater than any of the other enantiomers present in the mixture, e.g., in an amount greater than 50%. In one aspect, one enantiomer will predominate to the extent of 90% or to the extent of 91%, 92%, 93%, 94%, 95%, 96%, 97% or 98% or greater.

In one preferred embodiment, the enantiomer that predominates in a composition comprising a compound of Formula 1 is the S-enantiomer of Formula 1. In another preferred embodiment, the enantiomer that predominates in a composition comprising a compound of Formula 2 is the R-enantiomer of Formula 2.

In a preferred embodiment of the present invention, the enantiomer that is present as the sole enantiomer or as the predominate enantiomer in a composition of the present invention is represented by Formula 3 or Formula 5, wherein X₁, X₂, X₃, X₄, X₅, R₁, R₂, R₃, and R₄ are defined as above, or by Formula 7 or Formula 8.

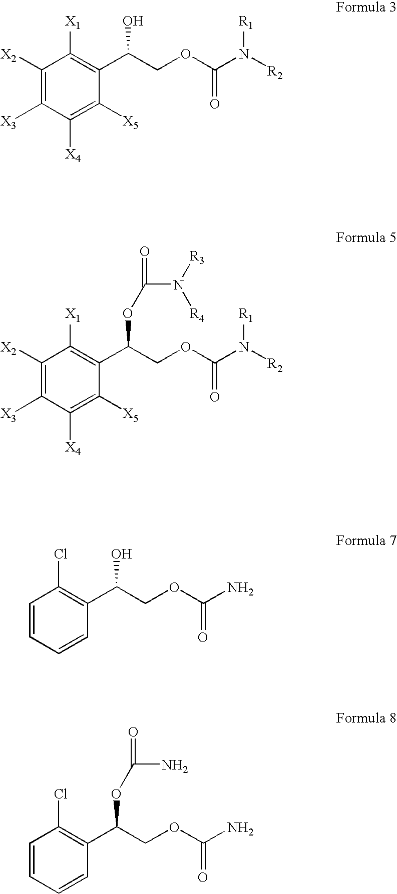

The present invention provides methods of using enantiomers

and enantiomeric mixtures of compounds represented by Formula 1 and Formula 2 or a pharmaceutically acceptable salt or ester form thereof:

A carbamate enantiomer of Formula 1 or Formula 2 contains an asymmetric chiral carbon at the benzylic position, which is the aliphatic carbon adjacent to the phenyl ring.

An enantiomer that is isolated is one that is substantially free of the corresponding enantiomer. Thus, an isolated enantiomer refers to a compound that is separated via separation techniques or prepared free of the corresponding enantiomer.

The term “substantially free,” as used herein, means that the compound is made up of a significantly greater proportion of one enantiomer. In preferred embodiments, the compound includes at least about 90% by weight of a preferred enantiomer.

In other embodiments of the invention, the compound includes at least about 99% by weight of a preferred enantiomer. Preferred enantiomers can be isolated from racemic mixtures by any method known to those skilled in the art, including high performance liquid chromatography (HPLC) and the formation and crystallization of chiral salts, or preferred enantiomers can be prepared by methods described herein.

Methods for the preparation of preferred enantiomers would be known to one of skill in the art and are described, for example, in Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972).

Additionally, compounds of the present invention can be prepared as described in U.S. Pat. No. 3,265,728 (the disclosure of which is herein incorporated by reference in its entirety and for all purposes), U.S. Pat. No. 3,313,692 (the disclosure of which is herein incorporated by reference in its entirety and for all purposes), and the previously referenced U.S. Pat. Nos. 5,854,283, 5,698,588, and 6,103,759 (the disclosures of which are herein incorporated by reference in their entirety and for all purposes).

The present invention is further directed to the treatment of Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) comprising administering to a subject in need thereof a therapeutically effective amount of a compound of Formula 1 or Formula 2 in combination with at least one additional psychoactive medication.

As used herein, the term “Pervasive Developmental Disorders (PDDs)” shall be defined to include; Autistic Disorder, Asperger's Disorder, Childhood Disintegrative Disorder (CDD), Rett's Disorder, and PDD-Not Otherwise Specified (PDD-NOS) as these disorders are described in the DSM-IV-TR (The Diagnostic and Statistical Manual of Mental Disorders, 4th edition Text Revision (DSM-IV-TR) (American Psychiatric Association, 2000)

As used herein, unless otherwise noted, the term “psychoactive medication” shall mean any pharmaceutical agent that may be used to treat or to augment the treatment of Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS). Suitable examples include, but are not limited to; stimulants, including but not limited to methylphenidate, amphetamine and modafinil, major tranquilizers, such as molindone, haloperidol and chlorpromazine, minor tranquilizers, including benzodiazepines, antidepressants, including but not limited to tricyclics such as imipramine, amitriptyline, desipramine, nortriptyline, doxepin, protriptyline, trimipramine, clomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, fluvoxamine, and the like; serotonin receptor antagonists such as nefazodone, and the like; combined serotonin-noradrenergic reuptake inhibitors such as venlafaxine, milnacipran and the like; noradrenergic and specific serotonergic agents such as mirtazapine, and the like; noradrenaline reuptake inhibitors such as reboxetine, and the like; atypical antidepressants such as bupropion, and the like; natural products such as Kava-Kava, St. John's Wort, and the like; dietary supplements such as s-adenosylmethionine, and the like, mono-amine oxidase inhibitors such as phenelzine, tranylcypromine, moclobemide, and the like. Preferably the stimulant is selected from the group consisting of methylphenidate and modafinil. Preferably, the antidepressant is selected from the group consisting of fluoxetine, paroxetine, citalopram, fluvoxamine, bupropion, venlafaxine and sertraline.

One skilled in the art would be able to readily determined recommended dosage levels for known and/or marketed psychoactive medications including antidepressant, tranquilizers, antipsychotic drugs and stimulant drugs by consulting appropriate references such as drug package inserts, FDA guidelines, the Physician's Desk Reference, and the like.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Wherein the present invention is directed to co-therapy or combination therapy, comprising administration of one or more compound(s) of Formula 1 or Formula 2 and one or more psychoactive medications, “therapeutically effective amount” shall mean that amount of the combination of agents taken together so that the combined effect elicits the desired biological or medicinal response. For example, the therapeutically effective amount of co-therapy comprising administration of a compound of formula (I) or formula (II) and at least on antidepressant would be the amount of the compound of formula (I) or formula (II) and the amount of the psychoactive medication that when taken together or sequentially have a combined effect that is therapeutically effective. Further, it will be recognized by one skilled in the art that in the case of co-therapy with a therapeutically effective amount, as in the example above, the amount of the compound of Formula 1 or Formula 2 and/or the amount of the psychoactive medication individually may or may not be therapeutically effective.

As used herein, the terms “co-therapy” and “combination therapy” shall mean treatment of a subject in need thereof by administering one or more compounds of Formula 1 or Formula 2 in combination with one or more additional psychoactive medication(s), wherein the compound(s) of Formula 1 or Formula 2 and the additional psychoactive medication(s) are administered by any suitable means, simultaneously, sequentially, separately or in a single pharmaceutical formulation. Where the compound(s) of Formula 1 or Formula 2 and the additional psychoactive medication(s) are administered in separate dosage forms, the number of dosages administered per day for each compound may be the same or different.

The compound(s) of Formula 1 or Formula 2 and the additional psychoactive medication(s) may be administered via the same or different routes of administration. Examples of suitable methods of administration include, but are not limited to, oral (po), intravenous (iv), intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of Formula 1 or Formula 2 and the additional psychoactive medication(s) may be administered according to simultaneous or alternating regimens, at the same or different times during the course of the therapy, concurrently in divided or single forms.

In an embodiment of the present invention is a method for the treatment of Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) comprising administering to a subject in need thereof a combination of one or more compounds of Formula 1 or Formula 2 with one or more compounds selected from the group consisting of stimulants, including but not limited to methylphenidate, amphetamine and modafinil, major tranquilizers, such as molindone, haloperidol and chlorpromazine, minor tranquilizers, including benzodiazepines, antidepressants, including but not limited to tricyclics such as imipramine, amitriptyline, desipramine, nortriptyline, doxepin, protriptyline, trimipramine, clomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, fluvoxamine, and the like; serotonin receptor antagonists such as nefazodone, and the like; combined serotonin-noradrenergic reuptake inhibitors such as venlafaxine, milnacipran and the like; noradrenergic and specific serotonergic agents such as mirtazapine, and the like; noradrenaline reuptake inhibitors such as reboxetine, and the like; atypical antidepressants such as bupropion, and the like; natural products such as Kava-Kava, St. John's Wort, and the like; dietary supplements such as s-adenosylmethionine, and the like, mono-amine oxidase inhibitors such as phenelzine, tranylcypromine, moclobemide, and the like.

Preferably, one or more compounds of Formula 1 or Formula 2 are administered in combination with one or more compounds selected from the group consisting of: stimulants, including but not limited to methylphenidate, amphetamine and modafinil; major tranquilizers, such as molindone, haloperidol and chlorpromazine, minor tranquilizers, including benzodiazepines, antidepressants, including but not limited to tricyclics such as imipramine, amitriptyline, desipramine, nortriptyline, doxepin, protriptyline, trimipramine, clomipramine, amoxapine, tetracyclics such as maprotiline, non-cyclics such as nomifensine; triazolopyridines such as trazodone; serotonin reuptake inhibitors such as fluoxetine, sertraline, paroxetine, citalopram, fluvoxamine, serotonin receptor antagonists such as nefazodone; combined serotonin-noradrenergic reuptake inhibitors such as venlafaxine, milnacipran, noradrenergic and specific serotonergic agents such as mirtazapine, and the like; noradrenaline reuptake inhibitors such as reboxetine, atypical antidepressants such as bupropion, natural products such as Kava-Kava, St. John's Wort, dietary supplements such as s-adenosylmethionine, and the like, mono-amine oxidase inhibitors such as phenelzine, tranylcypromine, moclobemide.

More preferably, one or more compounds of Formula 1 or Formula 2 are administered in combination with one or more compounds selected from the group consisting of stimulants, major tranquilizers, mono-amino oxidase inhibitors, tricyclics and serotonin reuptake inhibitors.

Most preferably, one or more compounds of Formula 1 or Formula 2 are administered in combination with one or more compounds selected from the group consisting of stimulants and serotonin reuptake inhibitors.

As used herein, unless otherwise noted, “halogen” shall mean chlorine, bromine, fluorine and iodine.

As used herein, unless otherwise noted, the term “alkyl” whether used alone or as part of a substituent group, includes straight and branched chains. For example, alkyl radicals include methyl, ethyl, propyl, isopropyl, butyl, isobutyl, sec-butyl, t-butyl, pentyl and the like. Unless otherwise noted, “lower” when used with alkyl means a carbon chain composition of 1-4 carbon atoms.

As used herein, unless otherwise noted, “alkoxy” shall denote an oxygen ether radical of the above described straight or branched chain alkyl groups, for example, methoxy, ethoxy, n-propoxy, sec-butoxy, t-butoxy, n-hexyloxy and the like.

As used herein, the notation “*” shall denote the presence of a stereogenic center.

When a particular group is “substituted” (e.g., alkyl, aryl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

Under standard nomenclature used throughout this disclosure, the terminal portion of the designated side chain is described first, followed by the adjacent functionality toward the point of attachment. Thus, for example, a “phenyl-alkyl-amino-carbonyl-alkyl” substituent refers to a group of the formula

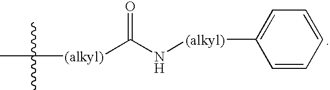

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following: acids, including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases, including; ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like.

Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular.

In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques.

For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above.

The pharmaceutical compositions herein will contain, per unit dosage unit, e.g., tablet, capsule, powder, injection, suppository, teaspoonful and the like, of from about 0.1-1000 mg and may be given at a dosage of from about 0.01-200.0 mg/kg/day, preferably from about 0.1 to 100 mg/kg/day, more preferably from about 0.5-50 mg/kg/day, more preferably from about 1.0-25.0 mg/kg/day or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, auto injector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof.

When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 1000 mg of the active ingredient of the present invention.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) described in the present invention may also be carried out using a pharmaceutical composition comprising any of the compounds as defined herein and a pharmaceutically acceptable carrier. The pharmaceutical composition may contain between about 0.1 mg and 1000 mg, preferably about 50 to 700 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings.

Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Compounds of this invention may be administered in any of the foregoing compositions and according to dosage regimens established in the art whenever treatment of Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) is required.

The daily dosage of the products may be varied over a wide range from 0.01 to 200 mg/kg per adult human per day. For oral administration, the compositions are preferably provided in the form of tablets containing, 25.0, 50.0, 100, 150, 200, 250, 400, 500, 600, 750 and 1000 milligrams of the active ingredient for the symptomatic adjustment of the dosage to the patient to be treated. An effective amount of the drug is ordinarily supplied at a dosage level of from about 0.1 mg/kg to about 200 mg/kg of body weight per day. Preferably, the range is from about 1.0 to about 20.0 mg/kg of body weight per day, more preferably, from about 2.0 mg/kg to about 15 mg/kg, more preferably, from about 4.0 to about 12.0 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails for the indication of Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS), including first-in-human, dose ranging and efficacy trials, in healthy patients and/or those suffering from a given disorder, may be completed according to methods well known in the clinical and medical arts.

### EXAMPLES

The following Examples are set forth to aid in the understanding of the invention, and are not intended and should not be construed to limit in any way the invention set forth in the claims which follow thereafter. All the examples below utilized one of the compounds of the invention. This compound is shown as Formula 7 above and will be referred to in the examples below as COMPOUND #7. The structure of COMPOUND #7 is shown below;

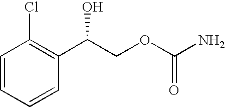

### Example 1

### Dominant-Submissive Rat In Vivo Assay

### Effects of COMPOUND #7 in the Dominant-Submissive Reaction, Animal Model of Mania and Depression (DD02313)

In this study, the effects of COMPOUND #7 on dominant or submissive behavior in pairs of rats competing for food are examined. It has been shown that anti-manic drugs, including anticonvulsants, decrease dominance and antidepressant drugs reduce submissiveness. This model uses dominant behavior as a model of mania and submissive behavior as a model of depression. The dominance and submissiveness is defined in a competition test and measured as the relative success of two food-restricted rats to gain access to a feeder. Rats are randomly paired and placed in an apparatus allowing them to compete for a food reward. The dominant-submissive relationship develops over a 2-week period. The submissive or dominant animals in pairs selected after 2 weeks of training were treated orally twice a week (b.i.d.) with COMPOUND #7 at 3 or 30 mg/kg for 5 weeks. The partner of the drug-treated animal was treated with vehicle.

A dose of 30 mg/kg of COMPOUND #7 increased competitiveness of both dominant and submissive rats. However, the effect of COMPOUND #7 on submissive rats was more extensive and had a faster onset. This effect was significant in submissive rats after the 1st week of treatment, while for dominant rats it was significant after the 2nd week of treatment. COMPOUND #7 at 3 mg/kg produced different effects in dominant and submissive rats. It decreased competitiveness of dominant rats and did not have an effect on submissive rats. The conclusion of the study was that COMPOUND #7 may act as an antidepressant at higher doses, and at lower doses, this agent may exhibit mood-stabilizing properties in acute mania.

The objective of this study was to determine if COMPOUND #7 is active in the Reduction of Submissive Behavior Model (RSBM) of depression and the Reduction of Dominant Behavior Model (RDBM) of mania. Measurements were made at two doses (3 and 30 mg/kg) after oral twice a day (b.i.d.) administration. The effect of the drug in the RSBM was compared to the effect of fluoxetine (10 mg/kg) and vehicle (0.5% methylcellulose). The effect of the drug in the RDBM was compared with the effect of lithium (100 mg/kg) and vehicle (0.5% methylcellulose). The endpoints measured were the development of a significant reduction of submissive or dominant behavior and its time of onset.

It has been shown that dominant behavior can serve as a model of mania and submissive behavior as a model of depression. (Malatynska E, et al. Reduction of submissive behavior in rats: a test for antidepressant drug activity. Pharmacology 2002; 64:8. and Malatynska E, et al. Dominant behavior measured in a competition test as a model of mania. In: International Behavioral Neuroscience Society Meeting, ed. IBNSCapri, Italy, 2002, p 26).

Treatment of the submissive subject for 3 weeks with imipramine, desipramine, or fluoxetine significantly and dose-dependently (fluoxetine) reduced submissive behavior. The effect was attenuated after cessation of treatment with desipramine. Treatment of submissive rats with the anxiolytic diazepam (See, Malatynska E, Goldenberg R, Shuck L, Haque A, Zamecki P, Crites G, Schindler N, Knapp R J. Reduction of submissive behavior in rats: a test for antidepressant drug activity. Pharmacology 2002; 64:8) or the psychostimulant amphetamine (unpublished observation) were ineffective.

Gardner has suggested that dominant behavior is related to mania (for a review on the relation of dominant-submissive behavior to mania and depression (See Gardner R Jr. Mechanisms in manic-depressive disorder: an evolutionary model. Arch Gen Psychiatry 1982; 39:1436)

We have shown that drugs commonly used to alleviate mania in the clinic such as lithium chloride, sodium valproate, carbamazepine, and clonidine significantly reduced competitive behavior when administered to dominant rats. (See, Malatynska E, Rapp R, Crites G. Dominant behavior measured in a competition test as a model of mania. In: International Behavioral Neuroscience Society Meeting, ed. IBNSCapri, Italy, 2002, p 26)

The onset of these effects for all drugs tested was similar to the onset of their therapeutic effect in patients. Thus, submissive behavior was sensitive to and selectively reduced by antidepressants. Dominant behavior was sensitive to a range of drugs used to treat mania in humans.

### Formation of Dominant-Submissive Relationship (DSR)

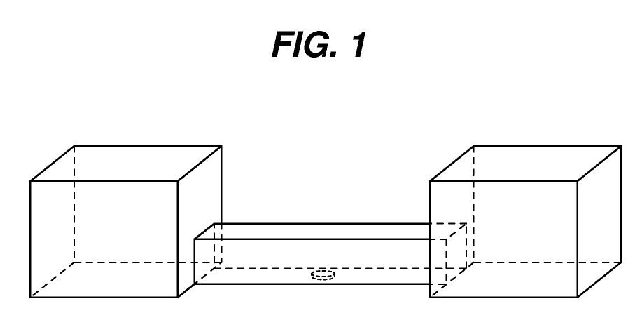

The DSR developed by two rats competing for food uses the apparatus in FIG. 1.

The methodology and equipment are described in several publications. (See: Malatynska E, Goldenberg R, Shuck L, Haque A, Zamecki P, Crites G, Schindler N, Knapp R J. Reduction of submissive behavior in rats: a test for antidepressant drug activity. Pharmacology 2002; 64:8; Malatynska E, Rapp R, Crites G. Dominant behavior measured in a competition test as a model of mania. In: International Behavioral Neuroscience Society Meeting, ed. IBNSCapri, Italy, 2002, p 26; Bonnet U. Moclobemide: therapeutic use and clinical studies. CNS Drug Rev 2003; 9:97; Danysz W, Plaznik A, Kostowski W, Malatynska E, Jarbe T U, Hiltunen A J, Archer T. Comparison of desipramine, amitriptyline, zimeldine and alaproclate in six animal models used to investigate antidepressant drugs. Pharmacol Toxicol 1988; 62:42; Knapp R J, Goldenberg R, Shuck C, Cecil A, Watkins J, Miller C, Crites G, Malatynska E. Antidepressant activity of memory-enhancing drugs in the reduction of submissive behavior model. Eur J Pharmacol 2002; 440:27; Kostowski W, Malatynska E, Plaznik A, Dyr W, Danysz W. Comparative studies on antidepressant action of alprazolam in different animal models. Pol J Pharmacol Pharm 1986; 38,471; Malatynska E, De Leon I, Allen D, Yamamura H I. Effects of amitriptyline on GABA-stimulated ³⁶Cl⁻ uptake in relation to a behavioral model of depression. Brain Res Bull 1995; 37:53; Malatynska E, Kostowski W. The effect of antidepressant drugs on dominance behavior in rats competing for food. Pol J Pharmacol Pharm 1984; 36:531 and Malatynska E, Kostowski W. Desipramine antagonizes clonidine-induced suppression of dominance in rats: possible involvement of amygdaloid nuclei. Pol J Pharmacol Pharm 1988; 40:357)

In the experiments described in this report Sprague-Dawley rats weighing 160 to 180 g were used. Testing for the development of a DSR between paired rats begins with the random assignment of rats into pairs. Rats from pairs are housed separately between test sessions with other animals in groups of four. The animals are food-deprived overnight with free access to water.

The test involves placing each member of a pair in opposite chambers of the testing apparatus. These chambers are connected through a narrow tunnel with a small container of sweetened milk at the center. Only one animal at the time can have comfortable access to the feeder. The test is conducted once a day over a 5-minute period and the time spent on the feeder by each animal is recorded. At the end of the 5-minute testing period the animals are separated, returned to their home cages and given free access to food (regular small laboratory animals chow) for a limited period of time (1 hour). The testing is suspended during weekends and the animals have free access to food during this time.

During the 1 st week (5 days) of testing, animals habituate to the new environment. During this 1st week (5 days) of testing the drinking scores vary considerably and these data are used only to detect any apparent reversals within the pairs of tested rats. Dominance is assigned to the animal with the highest score during the 2nd week of testing if three criteria are achieved. First, there must be a significant difference (two-tailed t-test, P <0.05) between the average daily drinking scores of both animals. Second, the dominant animal score must be at least 40% greater than the submissive animal's score. Third, there should be no reversals during the 2-week observation process. About 25% of the initial animal pairs achieve these criteria. Only these selected pairs are continued in the study for the next 3 to 6 weeks.

Table 1 shows the time necessary and number of animals required completing one experimental unit for studying either one drug at one dose or one animal strain, to have sufficient results for valid statistical analysis. The number of animals shown in the table is typical for manual scoring.

TABLE 1
Timetable for Basic Experimental Unit

| | | | | N of Pairs |
|---|---|---|---|---|
| | | | N of Animals | with D/S |
| Procedure | Time | N of Animals | Selected | Relation |
| 1st Week | 5 day | 32 | | |
| (habituation) | | | | |
| 2nd Week | 5 day | 32 | 10-14 | 5-7 |
| (selection) | | | | |
| Drug | 3-6 | 10-14 | | 5-7 |
| Administration | week | | | |

D/S = dominance/submissive
N = number of animals

### Drug Treatment

COMPOUND #7 was evaluated in the Rat Reduction of Submissive

Behavior Model (RSBM) of depression (Malatynska, E., Rapp, R., Harrawood, D., and Tunnicliff, G., Neuroscience and Biobehavioral Review, 82 (2005) 306-313; Malatynska, E., and Knapp, R. J., Neuroscience and Biobehavioral Review, 29 (2005) 715-737).

In the experiments described in this report five submissive rats were treated b.i.d., p.o. with COMPOUND #7 at 3 mg/kg and another five submissive rats were treated with COMPOUND #7 at 30 mg/kg for 5 weeks. The dominant rats from all these pairs were treated (b.i.d., p.o.) with vehicle (0.5% methylcellulose). The data were compared to the results from our previous experimental set where submissive rats were treated intraperitoneally (i.p.) once a day with fluoxetine (10 mg/kg) and dominant rats from these pairs were treated with vehicle (water), n=6.

In a separate set of experiments five dominant rats from two sets of paired animals were treated (b.i.d., p.o.) with either 3 or 30 mg/kg COMPOUND #7 for 5 weeks. The submissive rats from these pairs were treated (b.i.d., p.o.) with vehicle (0.5% methylcellulose). The data were compared to the results from our previous experimental set where dominant rats were treated i.p. with lithium chloride (100 mg/kg) and submissive rats from these pairs were treated with vehicle (water), n=4.

There was a control group for both sets of experiments with COMPOUND #7 to show the stability of the DSR where both rats from the pair, dominant and submissive, were treated with 0.5% methylcellulose, n=8.

### Data Processing and Statistical Analysis

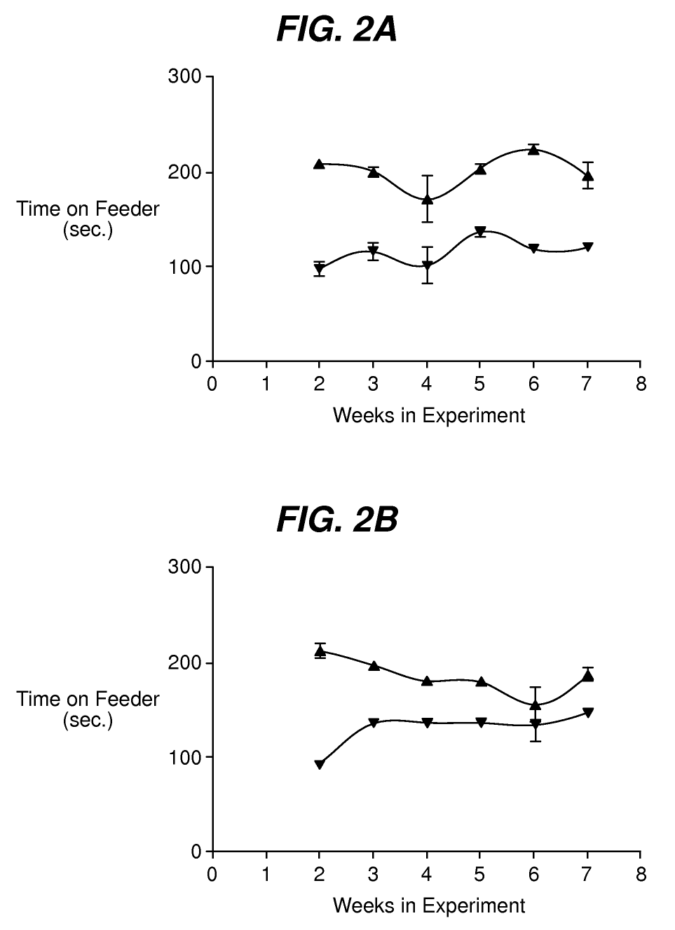

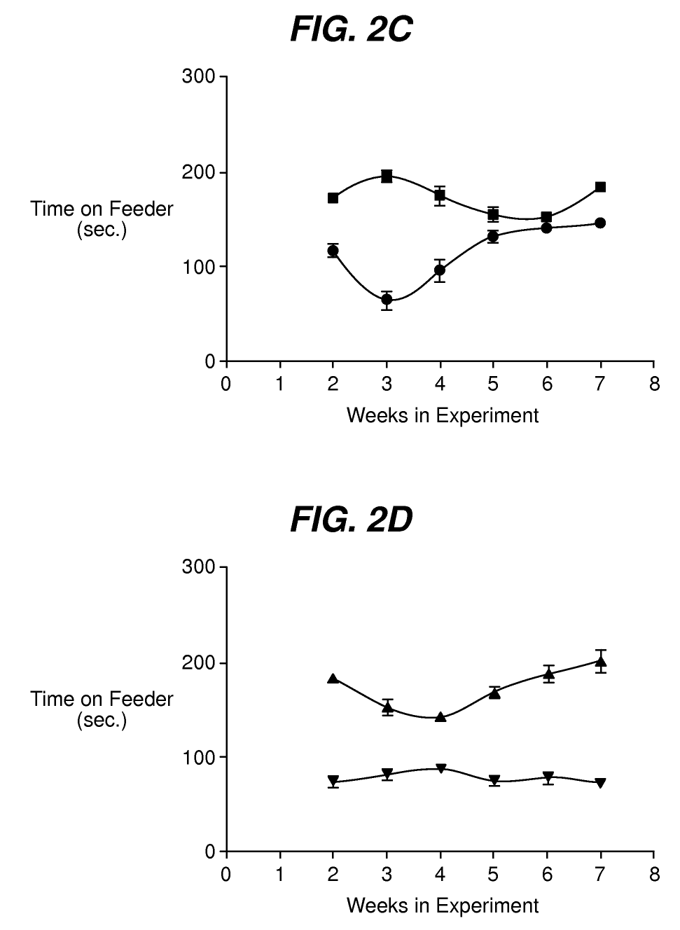

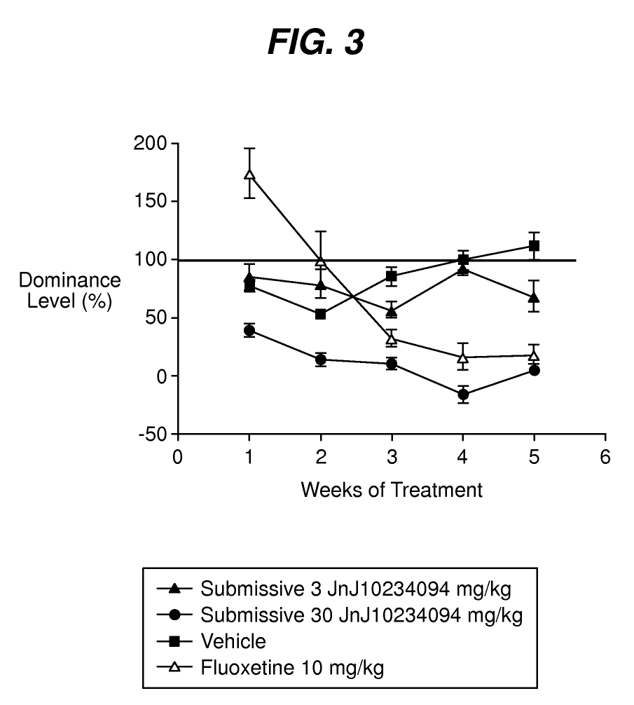

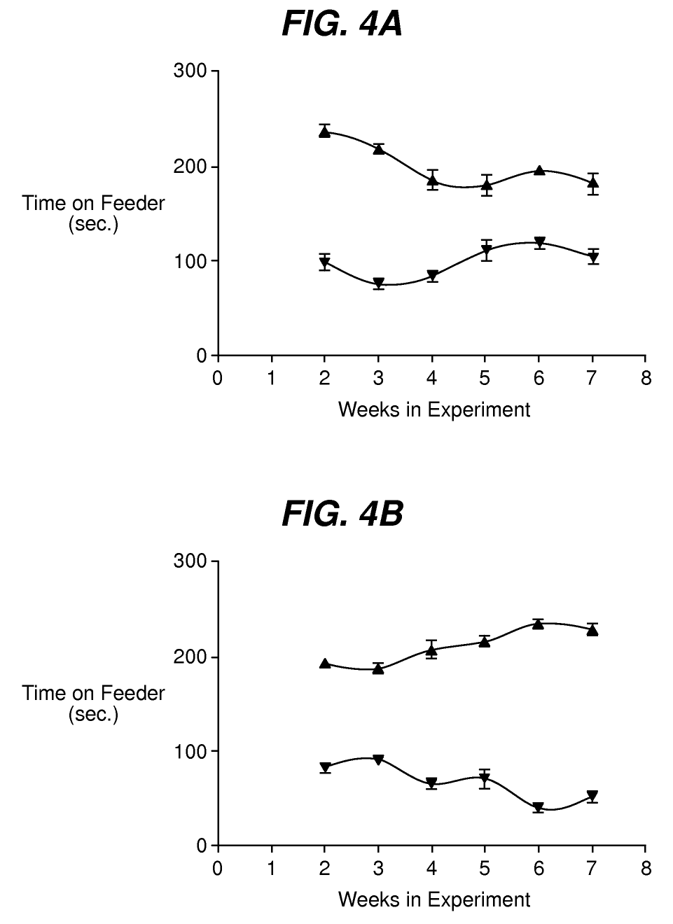

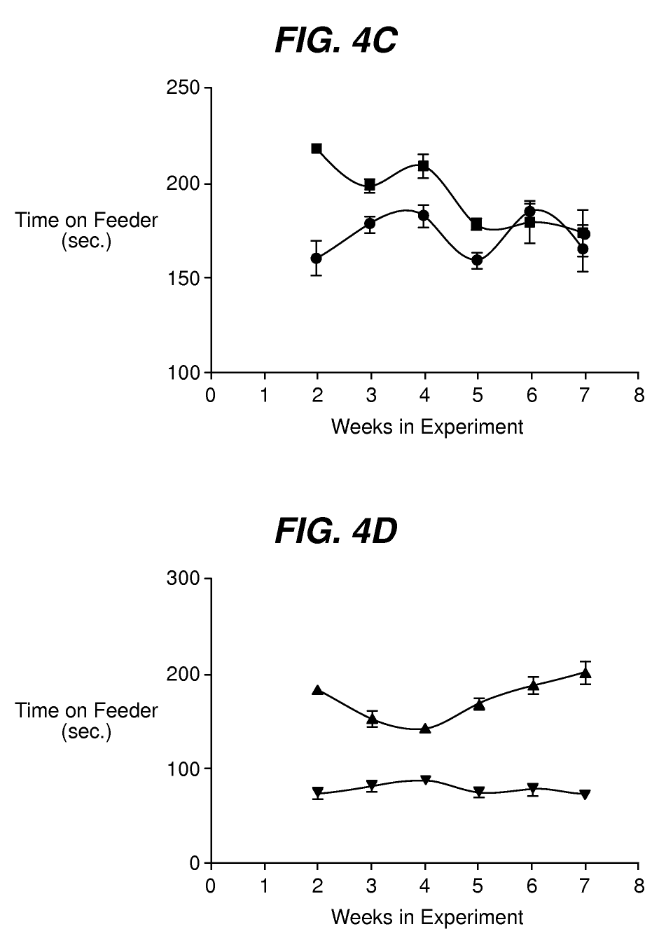

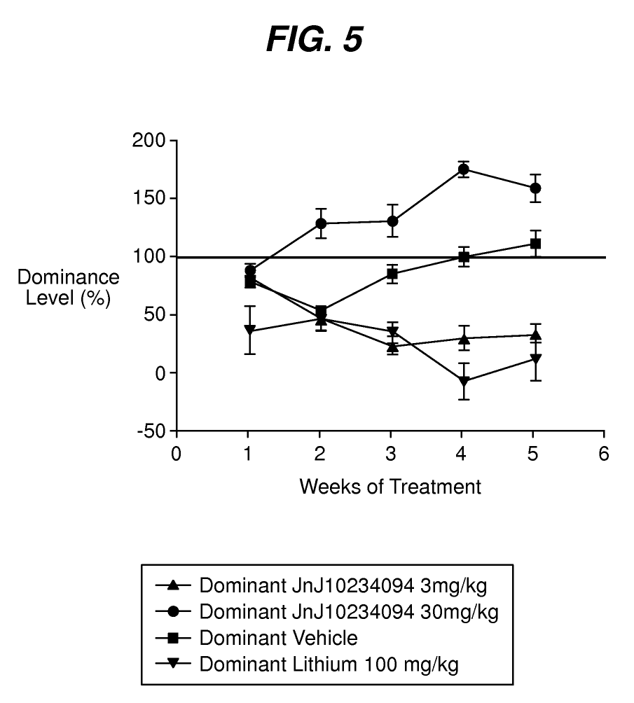

Endpoint measured in these experiments was time spent on feeder by individual rats from the pair during 5-minute daily session. Then, the average from the week was calculated (FIGS. 2 and 4). The treatment effect is often better captured as dominance level of the pair, because performance of the vehicle-treated paired rat is to some extent dependent on the performance of drug-treated rat. Dominance level is defined as the difference in averaged daily drinking scores for a 5-day week and reflects behavior of both animals in pair. The level of performance for different pairs of dominant and submissive rats may vary in the 2nd week of the study so the data for all rats were normalized to this initial week level (FIGS. 3 and 5). Thus, % of dominance level was calculated according the formula % DL=(T_{D}−T_{S}) week n×100/(T_{D}−T_{S}) week 2 with DL=dominance level, T_{D}=time spent by dominant rat, T_{S}=time of submissive rat, week n=test week n, week 2 (FIGS. 2 and 4) or 0 (FIGS. 3 and 5)=initial (selection) week.

The significant difference in the time spend on feeder by paired rats was calculated using two tailed t-test (Microsoft Excel). The significant differences between time spent on feeder by rats treated with different drugs were determined by analysis of variance (ANOVA) followed by Bonferroni multiple comparisons test using GraphPad Prism software (GraphPad Prism Software, Inc., San Diego, Calif.).

FIGS. 2 and 4 show data representing performance of paired dominant and submissive rats in the food competition test. In the experiment depicted in FIGS. 2A and 2B submissive rats and on FIGS. 4A and 4B dominant rats were treated with 3 or 30 mg/kg of COMPOUND #7. The respective partner rats were always treated with vehicle. The positive and negative control data are shown on panels C and D of FIGS. 2 and 4. The positive control for submissive rat treatment was provided by the serotonin reuptake inhibitor, fluoxetine (10 mg/kg, FIG. 2C) and for dominant rat treatment with the antimanic drug, lithium (100 mg/kg, FIG. 4C). Dominant and submissive rats in the pair treated simultaneously with vehicle provided negative controls for both experimental sets (FIGS. 2D and 4D). The dependent variable in these experiments was time spent on the feeder in seconds (y axis) and the independent variable was duration of the experiments in weeks (x axis). The habituation week data are omitted. The data plotted start on the 2nd week referred to as the initial week or selection week. In this week the performance of all dominant and submissive rats are significantly different. This significance is lost if the treatment has an effect or remains stable if the treatment does not have an effect.

It should be noted from FIGS. 2 and 4 that the drug mostly affects treated animal, observed as increased competitiveness of submissive rat treated with antidepressant or decreased competitiveness of dominant rat treated with antimanic drug. The transformed data as described in the Methods section (3.3) are presented in FIGS. 3 and 5. The dominance level of the initial week is marked as 100% for Week 0, before treatment week. The values of dominance level in the following after treatment weeks, 1-5, (x axis) are presented as data transformed according to the above discussed formula, (Methods section, 3.3). The data are presented on FIG. 3 for treatment of submissive rat from the pair and on FIG. 5 for treatment of dominant rat from the pair. This comparison confirms the effects observed in raw data and facilitates comparison of treatment effects.

### Effects of Compound #7 on Submissive Rats

COMPOUND #7 at 3 mg/kg did not have any effect on submissive rat behavior, similar to vehicle-treated submissive rats (FIGS. 2A and 2D). However, at the higher dose (30 mg/kg), COMPOUND #7 significantly increased competitiveness of submissive rat (FIGS. 2B and 3) compared to vehicle-treated submissive rats on the level of the corresponding week (FIGS. 2D and 3). This was similar to fluoxetine-treated submissive rats. COMPOUND #7 (FIGS. 2C and 3).

Thus, COMPOUND #7 has the same efficacy as fluoxetine but the onset of this effect was faster. The COMPOUND #7 (30 mg/kg) increased competitiveness of submissive rats after 1 week of treatment while the fluoxetine effect was only significant after 3 weeks of treatment.

### Effects of Compound #7 on Dominant Rats

COMPOUND #7 at 3 mg/kg decreased the performance of dominant rats (FIGS. 4A and 5). This effect was significant after 3 weeks of treatment. The extent and onset of the effect was not significantly different than the effect of lithium (FIGS. 4C and 5). At the higher dose (30 mg/kg) COMPOUND #7 significantly increased the competitiveness of dominant rats (FIG. 4B) as compared to water treated dominant rats (FIGS. 4D and 5). This effect was opposite the effect of lithium and COMPOUND #7's effect at the 3 mg/kg dose level. The onset of this effect occurred after 2 weeks of treatment.

The major finding of this study is that COMPOUND #7 affects the competitive behavior of both dominant and submissive rats. Effects of COMPOUND #7 to decrease dominant behavior and to increase competitiveness of submissive rats occurred at different doses. While dominant behavior was reduced at 3-mg/kg dose, the reduction of submissive behavior was most pronounced at 30 mg/kg. The 30-mg/kg dose increased competitiveness of both dominant and submissive rats. However, the effect of COMPOUND #7 on submissive rats was more extensive and with a faster onset. This effect was significant in submissive rats after the 1st week of treatment, while for dominant rats it was significant only after the 4th week of treatment. Because dominant behavior of competing rats was shown to model mania and submissive behavior was shown to model depression (See, Malatynska E, Goldenberg R, Shuck L, Haque A, Zamecki P, Crites G, Schindler N, Knapp R J. Reduction of submissive behavior in rats: a test for antidepressant drug activity. Pharmacology 2002; 64:8 and Malatynska E, Rapp R, Crites G. Dominant behavior measured in a competition test as a model of mania. In: International Behavioral Neuroscience Society Meeting, ed. IBNSCapri, Italy, 2002, p 26) it is possible that COMPOUND #7 may have mood stabilizing activity in both phases of bipolar disorders, depression, and mania.

Dominant-submissive behavior between animals can model human mood disorders. Submissive behavior has features of human depression that can be modeled using rats or mice in a behavioral paradigm referred to as the RSBM in which submissive behavior is reduced by antidepressant drugs. An analogous approach referred to as RDBM is sensitive to drugs used to treat mania. Neither model, RDBM or RSBM, is a complete model of bipolar disorder but they can be used together to model individual poles of bipolar symptoms. At this time the RSBM is better established than the RDBM. The studies confirming the validity of RDBM model should be extended. This study shows clearly that rats with different behavioral traits react differently to the same anticonvulsant agent. This is an important finding since diverse response to treatment occurs also in the clinic. Only about 40 to 70% of manic or depressive patients respond to a given antimanic or antidepressant drug, and the reason for this limitation is not known. Further work with this model could shed light on the mechanisms of the resistance to treatment.

We conclude that COMPOUND #7 dose dependently increases competitiveness of submissive rats therefore may act as an antidepressant. COMPOUND #7 at lower dose reduces dominant rat behavior. Thus, this agent may exhibit mood-stabilizing properties in acute mania at lower dose.

### REFERENCES

1. Malatynska E, Goldenberg R, Shuck L, Haque A, Zamecki P, Crites G, Schindler N, Knapp R J. Reduction of submissive behavior in rats: a test for antidepressant drug activity. Pharmacology 2002; 64:8.
2. Malatynska E, Rapp R, Crites G. Dominant behavior measured in a competition test as a model of mania. In: International Behavioral Neuroscience Society Meeting, ed. IBNSCapri, Italy, 2002, p 26.
3. Gardner R Jr. Mechanisms in manic-depressive disorder: an evolutionary model. Arch Gen Psychiatry 1982; 39:1436.
4. Ernst C L, Goldberg J F. Antidepressant properties of anticonvulsant drugs for bipolar disorder. J Clin Psychopharmacol 2003; 23:182.
5. Carpenter L L, Leon Z, Yasmin S, Price L H. Do obese depressed patients respond to topiramate? A retrospective chart review. J Affect Disord 2002; 69:251.
6. McElroy S L, Zarate C A, Cookson J, Suppes T, Huffman R F, Greene P, Ascher J. A 52-week, open-label continuation study of lamotrigine in the treatment of bipolar depression. J Clin Psychiatry 2004; 65:204.
7. Bonnet U. Moclobemide: therapeutic use and clinical studies. CNS Drug Rev 2003; 9:97.
8. Danysz W, Plaznik A, Kostowski W, Malatynska E, Jarbe T U, Hiltunen A J, Archer T. Comparison of desipramine, amitriptyline, zimeldine and alaproclate in six animal models used to investigate antidepressant drugs. Pharmacol Toxicol 1988; 62:42.
9. Knapp R J, Goldenberg R, Shuck C, Cecil A, Watkins J, Miller C, Crites G, Malatynska E. Antidepressant activity of memory-enhancing drugs in the reduction of submissive behavior model. Eur J Pharmacol 2002; 440:27.
10. Kostowski W, Malatynska E, Plaznik A, Dyr W, Danysz W. Comparative studies on antidepressant action of alprazolam in different animal models. Pol J Pharmacol Pharm 1986; 38,471.
11. Malatynska E, De Leon I, Allen D, Yamamura Hi. Effects of amitriptyline on GABA-stimulated ³⁶Cl⁻ uptake in relation to a behavioral model of depression. Brain Res Bull 1995; 37:53.
12. Malatynska E, Kostowski W. The effect of antidepressant drugs on dominance behavior in rats competing for food. Pol J Pharmacol Pharm 1984; 36:531.
13. Malatynska E, Kostowski W. Desipramine antagonizes clonidine-induced suppression of dominance in rats: possible involvement of amygdaloid nuclei. Pol J Pharmacol Pharm 1988; 40:357.

### Example 2

### Effects of Compound #7 in the Isolation Induced Aggression Model

The test compound used in this report is the same as that used in Example 1 and referred to therein as COMPOUND #7 shown as Formula #7 in the specification of this patent application. As discussed in detail below, data from the experiment showed that the test compound administered at doses of 40-mg/kg p.o. inhibited isolation-induced aggressive behavior in pairs of mice tested one hour after administration. This anti-aggressive effect of the test compound was not related to sedation.

The pharmacological effect shown by the test compound in inhibiting isolation induced aggression in this animal model suggests that this compound would have a beneficial effect on aggressiveness in humans and could improve impulse control and therefore is likely to be useful as a treatment for Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) in humans

The effects of test compound on the aggressive behavior of Crl:CD-1(ICR)BR albino mice were evaluated using the paradigm of isolation-induced aggression.

Test compound at 40-mg/kg p.o. statistically significantly inhibited isolation-induced aggressive behavior in pairs of mice tested 1 hour after administration (p<0.05). Test compound at 20-mg/kg p.o. also significantly shortened the onset of initiation of fighting in the treated group compared to the corresponding vehicle-treated group 4 hours after administration. The effects of test compound on general behavior were evaluated by visual observation using a checklist of behaviors. No behavioral or physical signs were observed in mice administered test compound at doses up to 100 mg/kg p.o., the test compound, administered at 300 mg/kg p.o., produced sedation. Results suggest that the anti-aggressive activity of test compound at 40-mg/kg p.o. is not related to sedation.

Male Crl:CD-1(ICR)BR albino mice, fasted overnight, were individually housed for 5 weeks in plastic cages on wood-chip bedding. Subsequently, they were paired for 1 minute daily for several days by placing one individually housed mouse (intruder) into the resident cage of another individually housed mouse (resident). The 1-minute pairing of intruder and resident mouse elicits aggressive behavior. Pairs of mice showing consistent aggressive behavior when paired for 1 minute, during several days were selected as subjects for drug testing.

Test compound (10 to 40 mg/kg p.o.) or the vehicle (methocel; aqueous 0.5%, w/v, hydroxypropyl methylcellulose solution) was administered (10 mL/kg) to the resident and intruder mouse. One and 4 hours after dosing, the mice were paired and the onset of fighting was recorded. Pairs of mice that did not fight within 1 minute were separated. The duration of fighting during a 1-minute test period was recorded. Mice were re-used in this procedure after several days to a week to allow metabolism and elimination of test compounds. The mice weighed 32 to 49 g at the time of testing and were fasted overnight before dosing. Mice were euthanized with CO₂ if they were sick or injured.

Results were expressed as the median onset of fighting and median duration of fighting in vehicle and drug-treated groups. The statistical significance of an increase in the median onset or a reduction in the median duration of fighting in pairs of mice given test compound or its vehicle 1 and 4 hours after administration was determined using the nonparametric Wilcoxon test (p<0.05, 1-tailed).

Test compound at 40-mg/kg p.o. inhibited isolation-induced aggressive behavior in pairs of mice tested 1 hour after administration, as shown by a statistically significant reduction (p <0.05; Wilcoxon rank sums, 1-tailed) in the median duration of fighting compared to that in the corresponding vehicle-treated group (See Tables 1A) When testing was repeated 4 hours after administration, the onset for initiation of fighting in the group that was administered test compound at 20 mg/kg p.o. was statistically significantly shorter than that in the corresponding vehicle-treated group. (See Table 1B)

The biological significance of this reduction in onset of fighting is unclear, because the median duration of fighting was not affected in any group that was administered test compound and tested 4 hours after administration compared to the corresponding vehicle-treated groups (See Tables 1A and 1B below).

In conclusion, the anti-aggressive activity of test compound at 40-mg/kg p.o. was not related to sedation, because no CNS-related effects were observed in other mice up to 4 hours after administration of test compound at 40 or 100-mg/kg p.o. although test compound at 300-mg/kg p.o. produced CNS-related effects in mice under the conditions of testing used. (See Table 2)

TABLE 1A
EFFECT OF TEST COMPOUND ON MEDIAN ONSET AND
DURATION OF ISOLATION-INDUCED FIGHTING
IN PAIRS OF MICE

| | | | 1 HOUR AFTER ADMINISRATION | | | |
|---|---|---|---|---|---|---|
| | | | Median | | Median | |
| | mg/kg | | Onset | | Duration | |
| Treatment | p.o. | N^{a} | (sec) | P | (sec) | P |
| Vehicle | 0 | 11 | 13 | — | 19 | — |
| Test Compound | 10 | 12 | 5 | 0.174 | 28 | 0.289 |
| Vehicle | 0 | 10 | 8 | — | 14 | — |
| Test Compound | 20 | 10 | 11 | 0.259 | 20 | 0.325 |
| Vehicle | 0 | 18 | 13 | — | 19 | — |
| Test Compound | 30 | 17 | 14 | 0.375 | 18 | 0.5 |
| Vehicle | 0 | 10 | 8 | — | 14 | — |
| Test Compound | 40 | 10 | 40 | 0.071 | 0.5^{b} | 0.035 |

^{a}Number of pairs of mice.
^{b}Compared to the vehicles treatment, run on the same day, the experimental compound produced either a statistically significant increase or decrease in onset or duration of fighting in paired mouse-isolates (p < 0.05; Willcoxon rank sums, 1-tailed).

TABLE 1B
EFFECT OF TEST COMPOUND ON MEDIAN ONSET AND
DURATION OF ISOLATION-INDUCED FIGHTING
IN PAIRS OF MICE

| | | | 4 HOURS AFTER ADMINISRATION | | | |
|---|---|---|---|---|---|---|
| | | | Median | | Median | |
| | mg/kg | | Onset | | Duration | |
| Treatment | p.o. | N^{a} | (sec) | P | (sec) | P |
| Vehicle | 0 | 11 | 34 | — | 2 | — |
| Test Compound | 10 | 12 | 4 | 0.092 | 6 | 0.366 |
| Vehicle | 0 | 10 | 50 | — | 12 | — |
| Test Compound | 20 | 10 | 4^{b} | 0.036 | 26 | 0.078 |
| Vehicle | 0 | 18 | 34 | — | 2 | — |
| Test Compound | 30 | 17 | 4 | 0.32 | 9.5 | 0.215 |
| Vehicle | 0 | 10 | 50 | — | 12 | — |
| Test Compound | 40 | 10 | 17 | 0.255 | 20 | 0.281 |

^{a}Number of pairs of mice.
^{b}Compared to the vehicles treatment, run on the same day, the experimental compound produced either a statistically significant increase or decrease in onset or duration of fighting in paired mouse-isolates (p < 0.05; Willcoxon rank sums, 1-tailed).

TABLE 2
EFFECTS OF ORAL ADMINISTRATION OF TEST COMPOUND
ON THE GENERAL BEHAVIOR OF MALE MICE

| | Number of Mice Affected | | | | | |
|---|---|---|---|---|---|---|
| | (N = 3/group) | | | | | |
| | Mg/kg p.o.^{a} | | | | | |
| | 40 | | 100 | | 300 | |
| Physical and Behavioral Signs | 1 h | 4 h | 1 h | 4 h | 1 h | 4 h |
| Decreased Open Field Activity | 0 | 0 | 0 | 0 | 3 | 2 |
| Impaired Horizontal Screen | 0 | 0 | 0 | 0 | 3 | 2 |
| Performance | | | | | | |
| Locomotor Ataxia | 0 | 0 | 0 | 0 | 0 | 2 |
| Soft Body Tone | 0 | 0 | 0 | 0 | 3 | 2 |
| Loss of Righting Reflex | 0 | 0 | 0 | 0 | 3 | 0 |
| Loss of Corneal Reflex | 0 | 0 | 0 | 0 | 3 | 0 |
| Loss of Pinnal Reflex | 0 | 0 | 0 | 0 | 3 | 0 |
| Extensor Limb Tone (hind limb) | 0 | 0 | 0 | 0 | 3 | 2 |
| Impaired Visual Placing Reflex | 0 | 0 | 0 | 0 | 3 | 0 |
| Decreased Startle Reflex | 0 | 0 | 0 | 0 | 3 | 0 |
| Reduced Skin Plasticity | 0 | 0 | 0 | 0 | 3 | 0 |
| Tail Pinch Response, Absent | 0 | 0 | 0 | 0 | 3 | 0 |
| Passive Response to Handling | 0 | 0 | 0 | 0 | 3 | 0 |

^{a}Test compound was suspended in the vehicle which consisted of 0.5%, w/v, aqueous hydroxypropyl methylcellulose, approximately 4000 cps; GFI-90000-000-E-004X, Lot 9428N.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A method for treating Pervasive Developmental Disorders (PDDs) including; Autistic Disorder, Asperger's Disorder, Childhood Disintegrative Disorder (CDD), Rett's Disorder, and PDD-Not Otherwise Specified (PDD-NOS) comprising administering to a subject in need thereof, a therapeutically effective amount of a compound of Formula 1 or Formula 2: 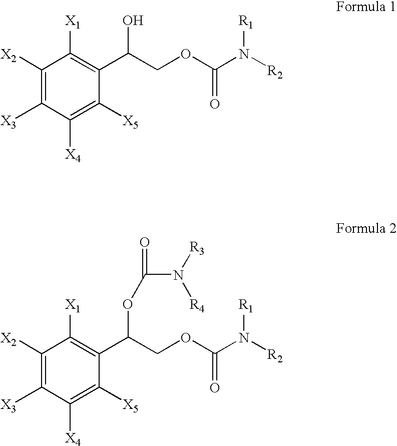
Or a pharmaceutically acceptable salt or ester form thereof
wherein:
R₁, R₂, R₃ and R₄ are independently hydrogen or C₁-C₄ alkyl,
wherein
C₁-C₄ alkyl is substituted or unsubstituted with phenyl, and
wherein
phenyl is substituted or unsubstituted with up to five substituents independently selected from; halogen, C₁₋C₄ alkyl, C₁₋C₄ alkoxy, nitro, cyano and amino
wherein amino is optionally mono or disubstituted with C₁₋C₄ alkyl,
and X₁, X₂, X₃, X₄ and X₅ are independently hydrogen, fluorine, chlorine, bromine or iodine.

2. The method of claim 1 wherein X is chlorine substituted at the ortho position of the phenyl ring and wherein R₁, R₂, R₃, R₄, R₅ and R₆ are selected from hydrogen.

3. A method for treating Pervasive Developmental Disorders (PDDs), comprising administering to a patient in need thereof a therapeutically effective amount of an enantiomer, or a pharmaceutically acceptable salt or ester thereof, selected from the group consisting of Formula (I) and Formula (II) or enantiomeric mixture wherein one enantiomer selected from the group consisting of Formula (I) and Formula (II) predominates: 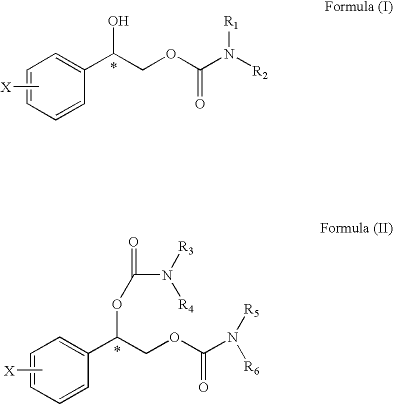
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

4. The method of claim 3 wherein X is chlorine substituted at the ortho position of the phenyl ring and wherein R₁, R₂, R₃, R₄, R₅ and R₆ are selected from hydrogen.

5. The method of claim 3 wherein one enantiomer selected from the group consisting of Formula (I) and Formula (II) predominates to the extent of about 90% or greater.

6. The method of claim 3 wherein one enantiomer selected from the group consisting of Formula (I) and Formula (II) predominates to the extent of about 98% or greater.

7. The method of claim 3 wherein the enantiomer selected from the group consisting of Formula (I) and Formula (II) is an enantiomer selected from the group consisting of Formula (Ia) and Formula (IIa): 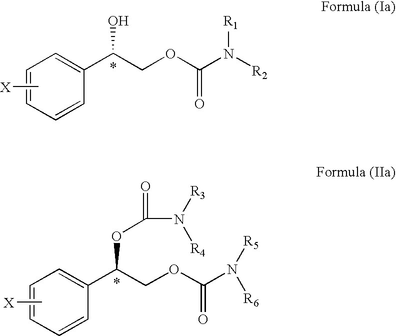
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl wherein phenyl is optionally substituted with substituents independently selected from the group consisting of halogen, C₁-C₄ alkyl, C₁-C₄ alkoxy, amino, nitro and cyano.

8. The method of claim 7 wherein X is chlorine substituted at the ortho position of the phenyl ring and wherein R₁, R₂, R₃, R₄, R₅ and R₆ are selected from hydrogen.

9. The method of claim 7 wherein one enantiomer selected from the group consisting of Formula (Ia) and Formula (IIa) predominates to the extent of about 90% or greater.

10. The method of claim 7 wherein one enantiomer selected from the group consisting of Formula (Ia) and Formula (IIa) predominates to the extent of about 98% or greater.

11. The method of claim 3 wherein the enantiomer selected from the group consisting of Formula (I) and Formula (II) is an enantiomer selected from the group consisting of Formula (Ib) and Formula (IIb) or a pharmaceutically acceptable salt or ester form thereof:

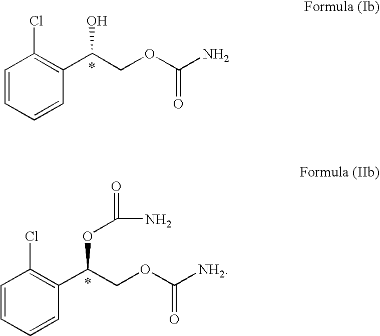

12. The method of claim 11 wherein one enantiomer selected from the group consisting of Formula (Ib) and Formula (IIb) predominates to the extent of about 90% or greater.

13. The method of claim 11 wherein one enantiomer selected from the group consisting of Formula (Ib) and Formula (IIb) predominates to the extent of about 98% or greater.

14. The method of claim 11 wherein the enantiomer is Formula (Ib) and predominates to the extent of 98% or greater.

15. The method of claim 1, wherein the Pervasive Developmental Disorders (PDDs) is Autistic Disorder.

16. The method of claim 1, wherein the Pervasive Developmental Disorders (PDDs) is Asperger's Disorder.

17. The method of claim 1, wherein the Pervasive Developmental Disorders (PDDs) is Childhood Disintegrative Disorder (CDD).

18. The method of claim 1, wherein the Pervasive Developmental Disorders (PDDs) is Rett's Disorder.

19. The method of claim 1, wherein the Pervasive Developmental Disorders (PDDs) is PDD-Not Otherwise Specified (PDD-NOS).

20. A method of treating Pervasive Developmental Disorders (PDDs) comprising administering to a subject in need thereof a therapeutically effective amount of an enantiomer selected from the group consisting of Formula (Ib) and Formula (IIb) that predominates to the extent of about 98% or greater.

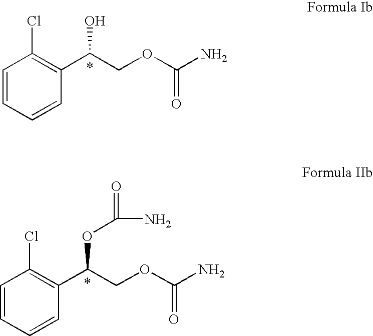

21. A method for the treatment of Pervasive Developmental Disorders (PDDs) comprising administering to a subject in need thereof a therapeutically effective amount of a compound of Formula Ib or a pharmaceutically acceptable salt thereof.

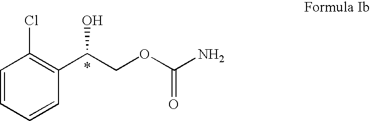

22. The method of claim 21, wherein the Pervasive Developmental Disorders (PDDs) is Autism.

23. A method for the treatment of Pervasive Developmental Disorders (PDDs) comprising administering, to a subject in need of co-therapy, a therapeutically effective amount of at least one additional psychoactive medication and a compound of Formula Ib or Formula IIb

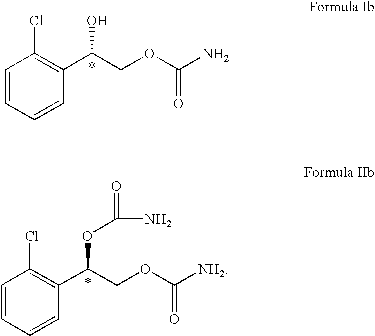

24. The method of claim 23 wherein the additional psychoactive medication is selected from the group consisting of; stimulants, including methylphenidate, amphetamine and modafinil, major tranquilizers, such as molindone, haloperidol and chlorpromazine, minor tranquilizers, including benzodiazepines, antidepressants, including but not limited to tricyclics such as imipramine, amitriptyline, desipramine, nortriptyline, doxepin, protriptyline, trimipramine, clomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, fluvoxamine, and the like; serotonin receptor antagonists such as nefazodone, and the like; combined serotonin-noradrenergic reuptake inhibitors such as venlafaxine, milnacipran and the like; noradrenergic and specific serotonergic agents such as mirtazapine, and the like; noradrenaline reuptake inhibitors such as reboxetine, and the like; atypical antidepressants such as bupropion, and the like; natural products such as Kava-Kava, St. John's Wort, and the like; dietary supplements such as s-adenosylmethionine, and the like, mono-amine oxidase inhibitors such as phenelzine, tranylcypromine, moclobemide.

25. A method for the treatment of Pervasive Developmental Disorders (PDDs) comprising administering to a subject in need thereof co-therapy with a therapeutically effective amount of at least one additional psychoactive medication and a compound of Formula Ib 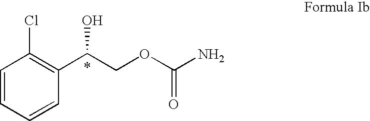
or a pharmaceutically acceptable salt thereof.
